SEQUENCE LISTING (5 sequences)

SEQ ID NO: 1 - protein, 13 aa, Homo sapiens
QLYENKPRRPYIL

SEQ ID NO: 2 - protein, 13 aa, Homo sapiens
  MISC_FEATURE (1)..(1): Pyro
ELYENKPRRPYIL

SEQ ID NO: 3 - protein, 10 aa, Homo sapiens
QHWSYGLRPG

SEQ ID NO: 4 - protein, 6 aa, Artificial Sequence
  synthetic peptide
DDHSVA

SEQ ID NO: 5 - protein, 6 aa, Homo sapiens
RRPYIL

(19) United States
(12) Patent Application Publication
(10) Pub. No.: US 2011/0319339 A1
(43) Pub. Date: Dec. 29, 2011

(54) NEUROTENSIN-DERIVED BRANCHED PEPTIDES AND USES THEREOF

(76) Inventors: Luisa Bracci, Siena (IT); Alessandro Pini, Siena (IT); Chiara Falciani, Siena (IT); Stefano Menichetti, Empoli (FL) (IT)

(21) Appl. No.: 13/127,346

(22) PCT Filed: Nov. 4, 2009
(86) PCT No.: PCT/EP2009/064619

§ 371 Date: May 9, 2011

Related U.S. Application Data
(60) Provisional application No. 61/111,970, filed on Nov. 6, 2008.

(51) Int. Cl. A61K 38/17; G01N 33/574; A61K 38/10; A61P 35/00; C07K 7/06; C07K 14/47

(52) U.S. Cl. 514/19.5; 530/327; 530/325; 530/324; 514/21.5; 514/21.4; 514/21.3; 514/19.2; 514/19.3; 435/7.23

(57) ABSTRACT

The present invention relates to a multimeric molecule having the general formula A or B:

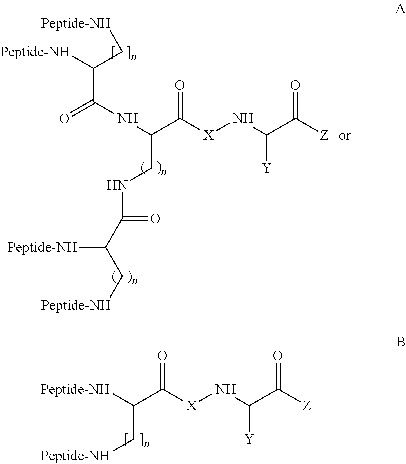

and its use in the diagnosis and/or therapy of tumors.

### FIELD OF THE INVENTION

The instant invention refers to in vivo stable branched peptides derived from the sequence of Neurotensin (NT). The peptides may be conjugated to functional units for specific targeting of cancer cells. Thus they can be used for diagnosis and/or therapy of tumors.

### BACKGROUND ART

One of the major problems in classic chemotherapy is the non-specific toxicity of most anticancer agents even for normal cells. Then, specific targeting of tumors is the main challenge in the research on cancer therapy and diagnosis.

Presently, innovative tumor-specific therapies follow the strategy of targeting tumor associated proteins, specifically expressed or over expressed on tumor cells.

The observation that receptors for different endogenous regulatory peptides are expressed in a number of primary human cancers, opened new perspectives on the use of synthetic peptides for tumor-selective targeting¹⁻³.

Neurotensin (NT) is a 13 amino acid peptide that has the dual function of neurotransmitter or neuromodulator in the central nervous system and local hormone in the periphery. NT receptors are overexpressed in severe malignancies such as small cell lung cancer, colon, pancreatic and prostate carcinomas. NT stabilized analogues have been proposed for tumor therapy several years ago⁴⁻¹⁰ and NT is still considered the best possible candidate for a peptide-based therapy of exocrine pancreatic carcinomas¹¹ in consideration of the high incidence and density of NT receptors in these tumors. Over 75% of all ductal pancreatic carcinomas over-express NT receptors, whereas normal pancreas tissue, pancreatitis and endocrine pancreas do not¹².

Tumor receptor-targeting is fundamental in approaching the problem of non-specific toxicity of cancer chemotherapies and it is a precious tool for tumor localization by radioisotopes. Nonetheless, the in vivo use of peptides has largely been limited by their short half-life.

The inventors of the present invention previously demonstrated that synthesis of peptides in branched dendrimeric form results in molecules that can retain peptide biological activity and are very resistant to proteolytic activity of biological fluids, thus having a markedly higher half-life with respect to monomeric peptides¹³⁻¹⁴.

The instant invention refers to in vivo stable dendrimeric peptides derived from NT. Such peptides are also conjugated to functional units to be used to target cancer cells. In particular, conjugation of NT4(8-13) with either the photosensitizer Che6 or the chemotherapic molecule MTX has shown to be specific for tumor cells and non-toxic to healthy ones, then overcoming the secondary effects of the classic chemotherapeutics when given systemically to mice¹⁵.

In the present invention, in addition to the specificity of the molecules, an increased activity of the conjugated molecules compared to free uncoupled drug was observed. The molecules of the present invention were selected among a pool of numerous analogues that the inventors synthesized. The ‘carrier’ peptide (neurotensin) is the best carrier among a number of others. For example, luteinizing hormone-releasing hormone (LHRH), also known as gonadotropin-releasing hormone (GnRH) (QHWSYGLRPG, SEQ ID No. 3), presented in vitro binding profiles lower than NT. In addition, the linkers used in the present invention were selected for each new molecule because of their large influence on the activity of the molecule. For example NT(8-13)4-beta-Ala-Biotin was shown to loose binding activity to NT receptor compared to NT(8-13)4; whereas NT4-peg-Biotin maintained IC50 values comparable to that of NT4. NT(1-13)4-Fluorescein and NT(8-13)4-Fluorescein are able to stain cancer cells and tissue more efficiently than NT(8-13)4-beta-Ala-K(PEG-Fluorescein) and the fluorophore is also more stable. Finally, the chemotherapeutic moiety was chosen on the basis of its functional groups such as to be selectively and univocally used for the coupling. The strength of the bond was modulated depending on the site and mode of action of the drug itself, the best conjugation was selected. As an example 6-mercaptopurine conjugated to neurotensin through an ‘uncleavable’ linker was completely non cytotoxic, the same drug conjugated through a ‘cleavable’ linker was active, but the activity resulted insufficient to be considered for further development. Similarly, monastrol derivative is not the first choice of compound in the present invention. Taken together, these specific features render the objects of the present invention unique and preferable over any possible analogue.

### SUMMARY OF INVENTION

It is an object of the present invention a multimeric molecule having the general formula A or B:

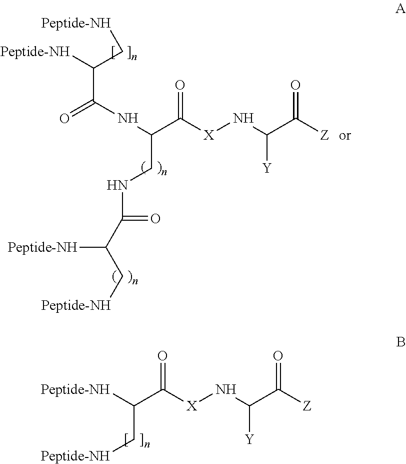

### Wherein

n=1-5,

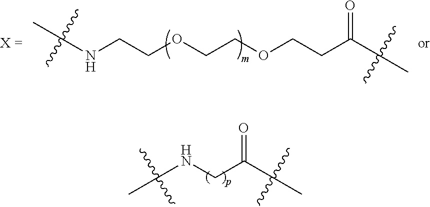

wherein m=9-12 and p=1-5,

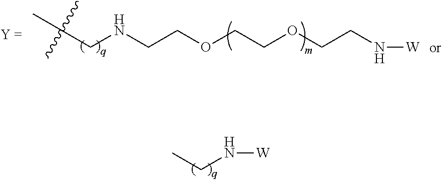

wherein q=1-5 and m=9-12;

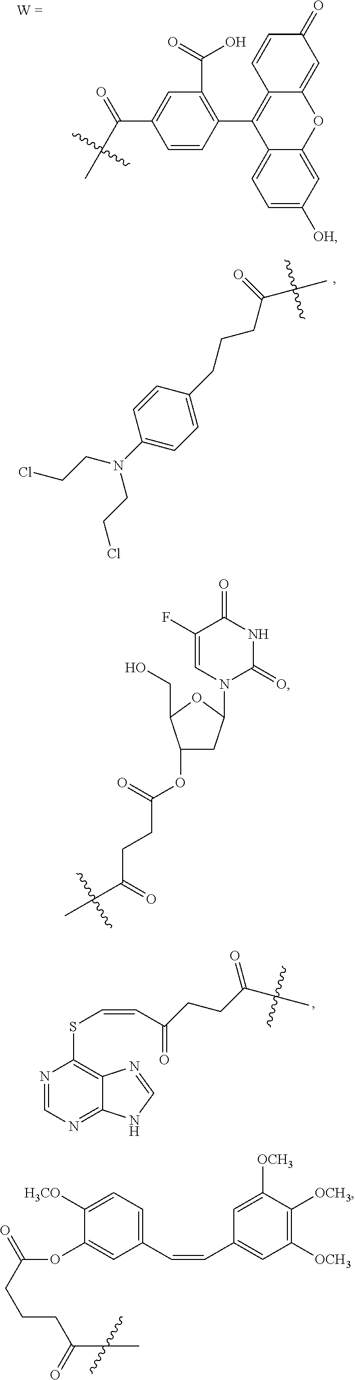

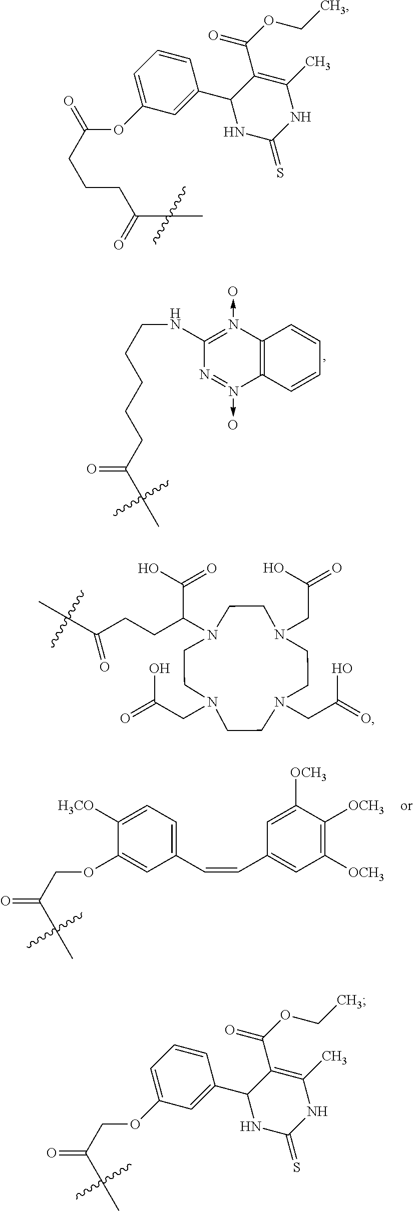

### and Z=NH₂ or OH.

Preferably the peptide comprises the following amino acid sequence: QLYENKPRRPYIL (Neurotensin, SEQ ID No. 1), pyroELYENKPRRPYIL (SEQ ID No. 2) or RRPYIL (Neurotensin 8-13, i.e. aa 8 to aa 13 of SEQ ID No. I).

Preferably, the multimeric molecule of the invention is for medical use.

Still preferably as anti-tumoral agent.

More preferably the tumour is a colon or pancreas or prostate carcinoma. It is a further object of the invention the use of the multimeric molecule of the invention for the preparation of a medicament.

Preferably the medicament has an anti-tumoral activity.

It is a further object of the invention the use of the multimeric molecule of the invention for tumor diagnostic.

Preferably the tumor is a colon or pancreas or prostate carcinoma. It is a further object of the invention a pharmaceutical composition comprising the multimeric molecule of the invention, or a pharmaceutically acceptable and efficient salt thereof and diluents, and/or solvents and/or carriers and/or excipients and/or vehicle.

It is a further object of the invention a method of treatment comprising the administration or exposure to the multimeric molecule of the invention.

Preferably the treatment is anti-tumoral.

It is a further object of the invention a method of diagnosis comprising the administration or exposure to the multimeric molecule of the invention.

Preferably the diagnosis is a tumour diagnosis. Still preferably the tumour is a colon or pancreas or prostate carcinoma.

The invention will be now described by non limiting examples referring to the following figures:

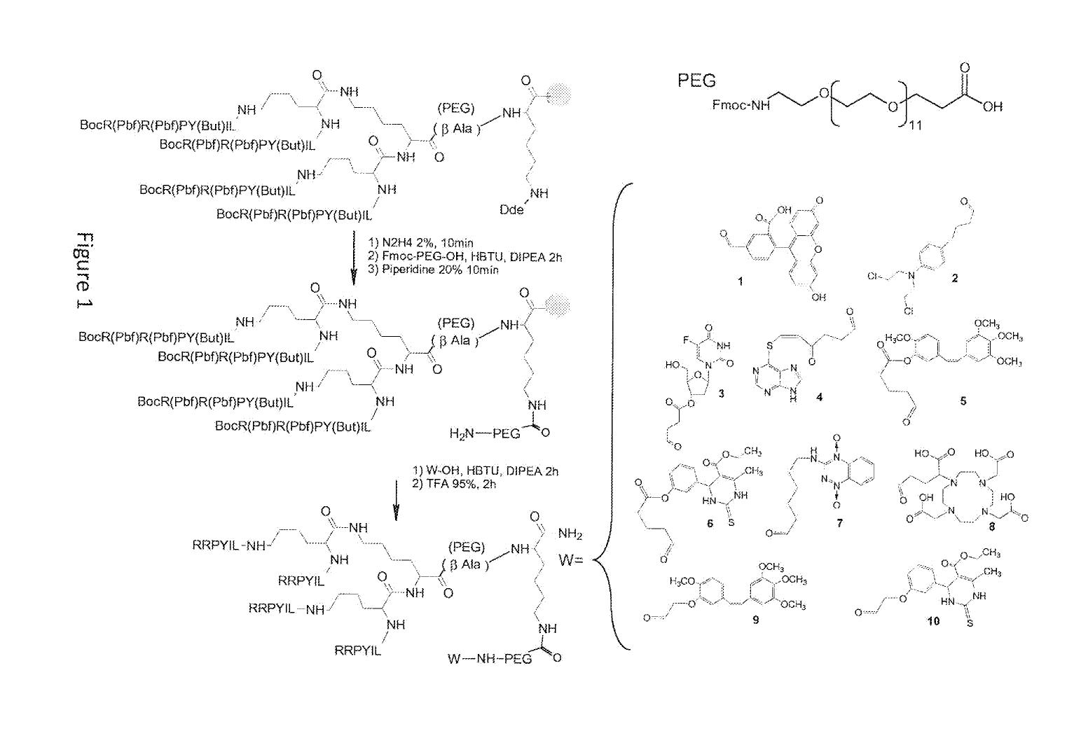

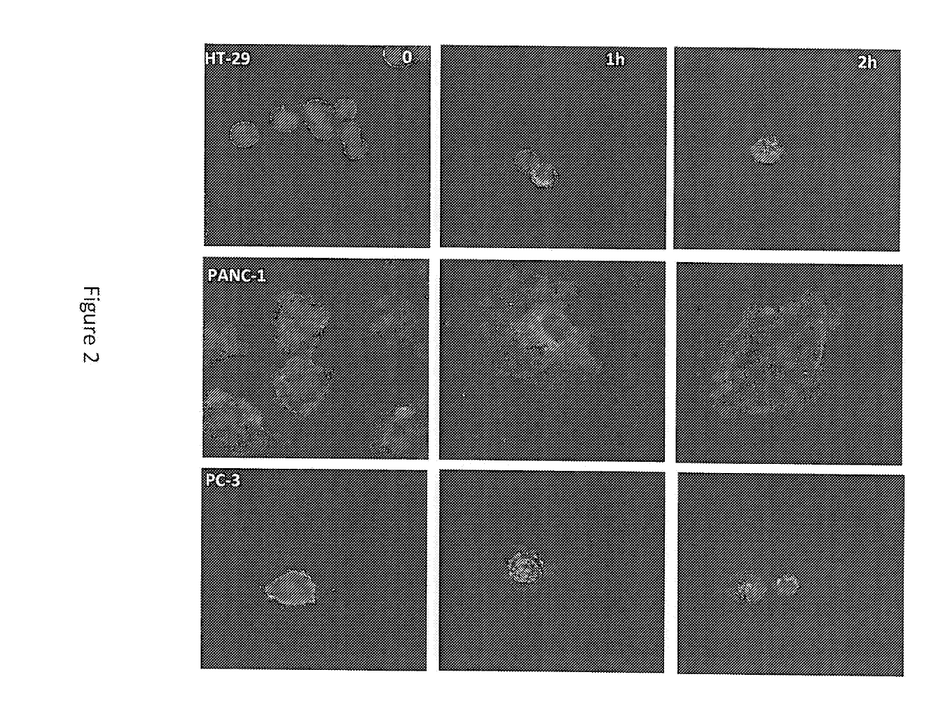

FIG. 1. Synthesis of tetrabranched NT(8-13)4-PEG-K(PEG-Fluorescein) [NT4(8-13)-FLUO] 1, NT(8-13)4-betaAla-K(PEG-Chlorambucil) [NT4(8-13)-CLB] 2, tetrabranched NT(8-13)-betaAla-K(PEG-5-fluorodeoxyuridine) [NT4(8-13)-5-FdU] 3, tetrabranched NT(8-13)-betaAla-K(PEG-6-mercaptopurine) [NT4(8-13)-6-MP] 4, tetrabranched NT(8-13)-betaAla-K(PEG-Combretastatin) [NT4(8-13)-CBTST] 5, tetrabranched NT(8-13)-betaAla-K(PEG-Monastrol) [NT4(8-13)-Mon] 6, tetrabranched NT(8-13)-betaAla-K(PEG-Tirapazamine) [NT4(8-13)-TPZ] 7, tetrabranched. NT(8-13)-betaAla-K(PEG-Asp-DOTA) [NT4(8-13)-DOTA] 8 as well as tetrabranched NT(8-13)-betaAla-K(PEG-Combretastatin ether) [NT4(8-13)-O—CBTST] 9, and tetrabranched NT(8-13)-betaAla-K(PEG-Monastrol ether) [NT4(8-13)-O-Mon] 10. NT(1-13)4 analogues carry the amino acid sequence pyroELYENKPRRPYIL (SEQ ID No. 2).

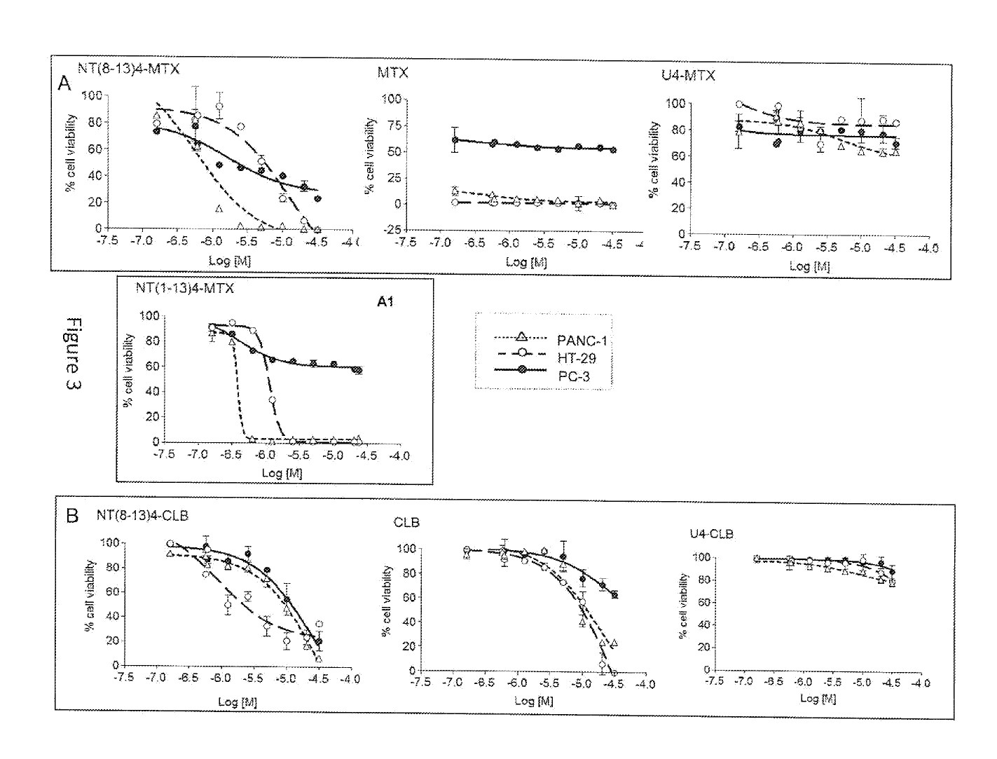

FIG. 2. Binding and internalization in tumor cell lines. HT-29, PC-3 and PANC-1 were exposed for 30 minutes (time 0) to NT(1-13)4-Fluorescein. Images were taken at time zero and after 1 and 2 hours of incubation in medium at 37° C. Cell membrane was stained with Lectin-Cy3 (red) and nuclei with DAPI (blue).

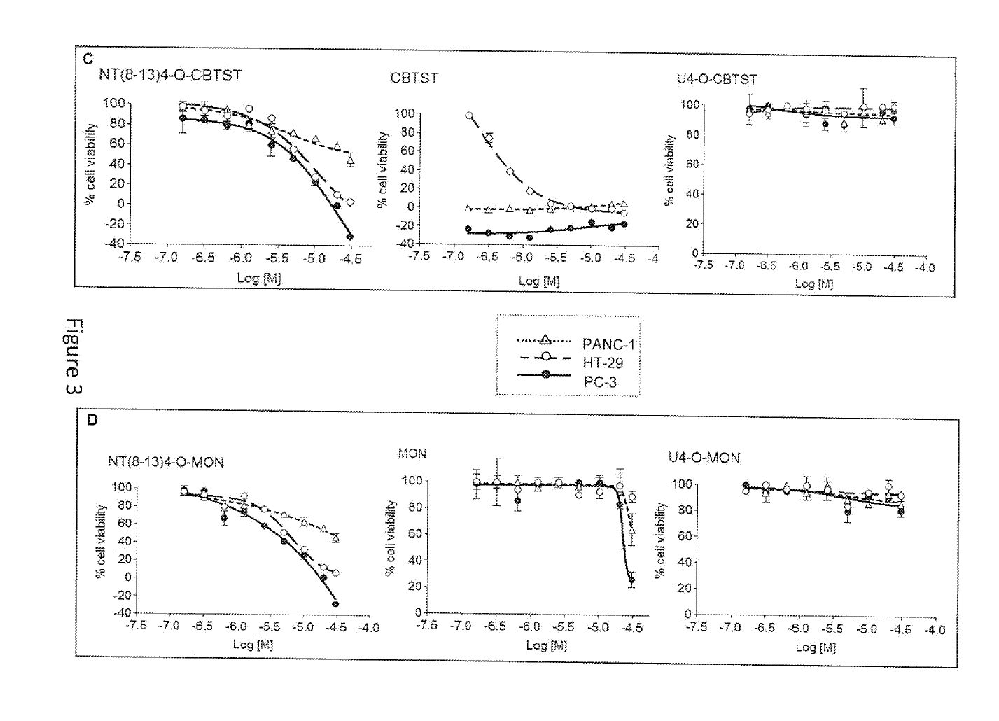

FIG. 3. Cytotoxicity of drug-conjugated slow-releasing branched NT on HT-29, PC-3 and PANC-1 cells. Panel A) From the left: NT(8-13) conjugated to methotrexate (MTX), free MTX; unrelated peptide (U4) conjugated to MTX, A1: NT(1-13) conjugated to MTX. Panel B) From the left: NT(8-13) conjugated to Chlorambucil—compound 2—(CLB), free CLB; unrelated peptide (U4) conjugated to CLB. Panel C) From the left: NT(8-13) conjugated to Combretastain-ether—compound 9—(O—CBTST); free CBTST; unrelated peptide (U4) conjugated to CBTST. Panel D) From the left: NT(8-13) conjugated to Monastrol-ether—compound 10—(MON), free MON; unrelated peptide (U4) conjugated to MON. Panel E) From the left: NT(8-13) conjugated to Tirapazamine—compound 7—(TPZ), free TPZ; unrelated peptide (U4) conjugated to TPZ.

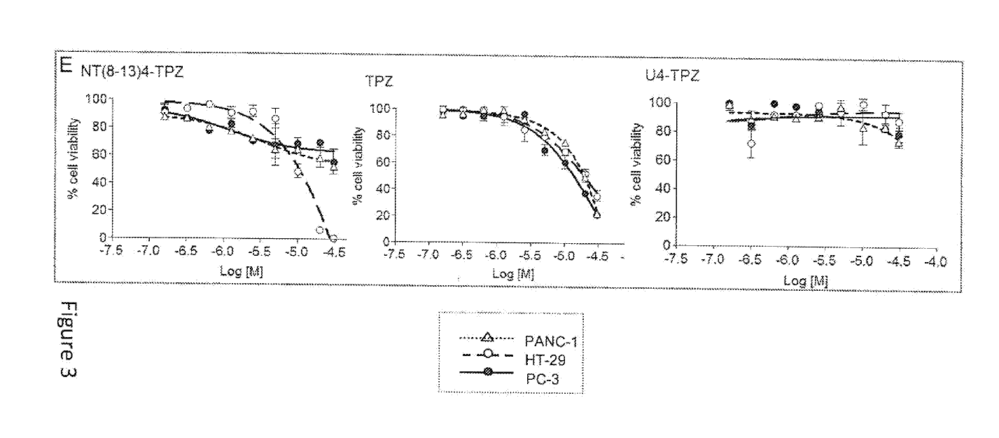

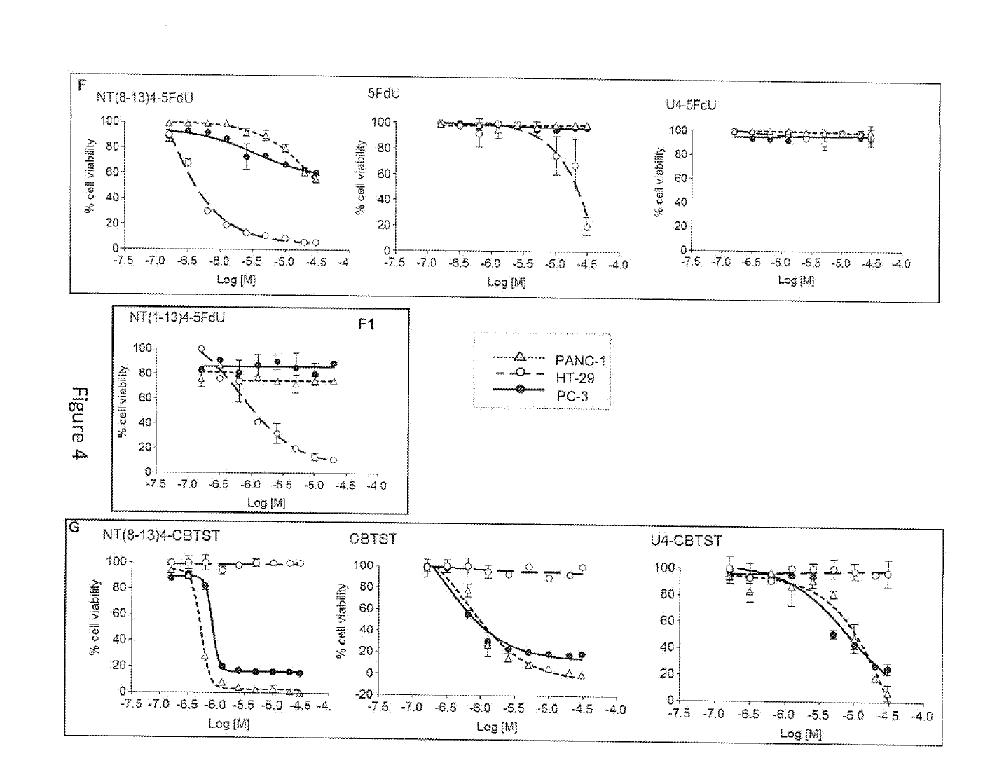

FIG. 4. Cytotoxicity of drug-conjugated fast-releasing branched NT on HT-29, PC-3 and PANC-1 cells. Panel F) From the left: NT(8-13) conjugated to 5-Fluorodeoxyuridine—compound 3—(5FdU), free 5FdU, unrelated peptide (U4) conjugated to 5FdU, F1: NT(1-13) conjugated to 5FdU. Panel. G) From the left: NT(8-13) conjugated to Combretastatin ester—compound 5—(CBTST), free CBTST, unrelated peptide, (U4) conjugated to CBTST.

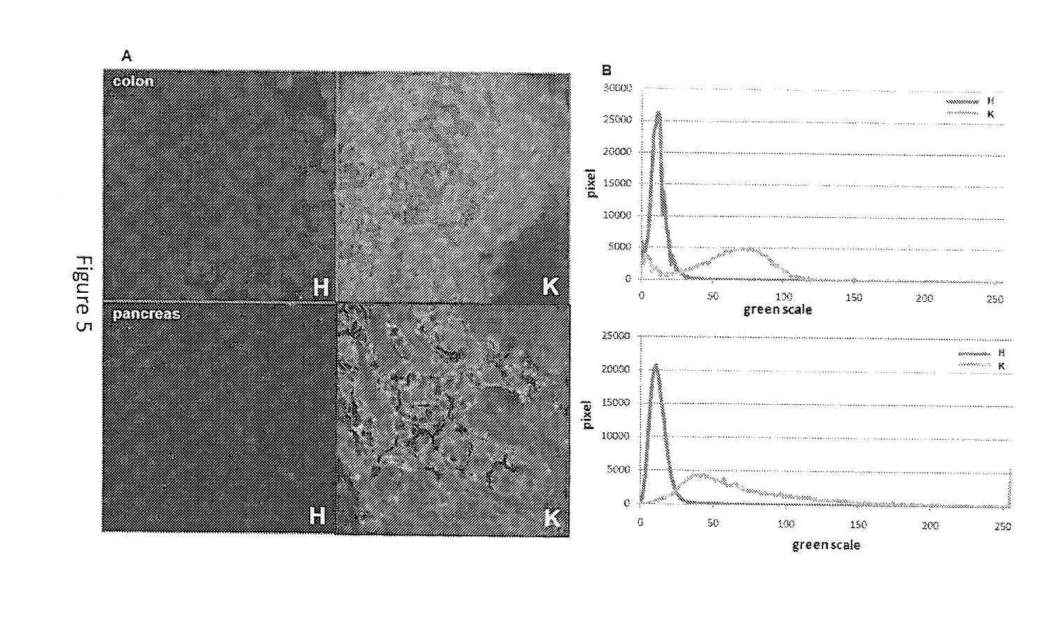

FIG. 5. Colon and pancreas adenocarcinoma (K) and corresponding healthy tissues (H) stained with NT(1-13)4-Fluorescein). Confocal microscopy images (A) were analyzed for pixel distribution with ImageJ software and were reported as pixel number in the green scale of the RGB system. (B) Comparison of mean fluorescence values in normal (light gray) and cancer (gray) samples for colon (n=17) and pancreas (n=12) human specimens.

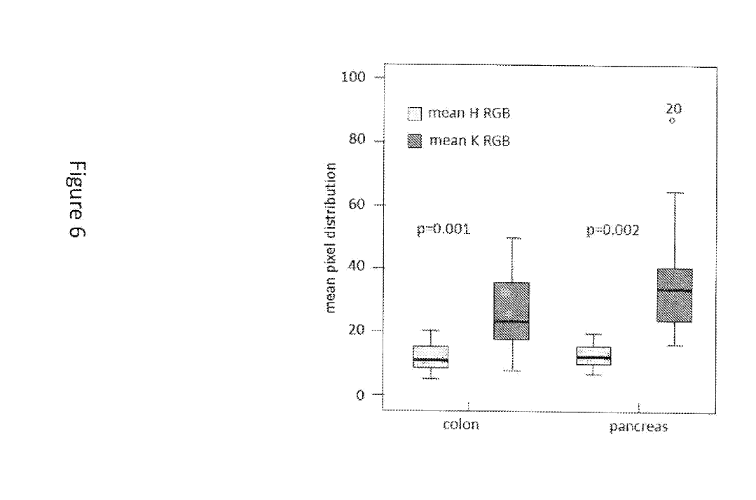

FIG. 6. Boxplot. The box for each category represents the interquartile range (25-75th percentile) and the black line within the box is the median value. Bottom and top bars of the whisker indicate the 10th and 90th percentiles, respectively. Outlier values are indicated with open circles; p values between the two groups are indicated.

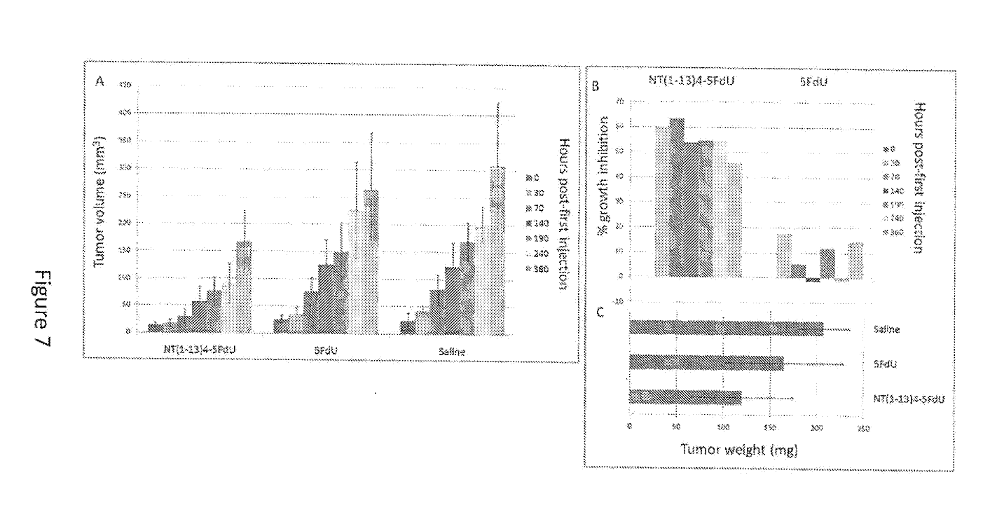

FIG. 7. Tumor growth reduction in mice. A, tumor volume of mice injected with NT(1-13)-5FdU (group 1), free 5FdU (group 2) and saline (group 3), B, inhibition of tumor growth calculated as a difference between tumor volumes of the untreated mice (group 3, considered 100%) and those treated with NT(1-13)-5FdU and free 5FdU (groups 1 and 2), C, tumor weight.

### DETAILED DESCRIPTION OF THE INVENTION

### Peptide Synthesis

Tetrabranched NT(8-13)-PEG-K(PEG_Fluorescein) [NT4(8-13)-Fluo] 1, NT(8-13)-PEG-Chlorambucil [NT4(8-13)-CLB] 2, tetrabranched NT(8-13)-PEG-5-fluorodeoxyuridine [NT4(8-13)-5-FdU] 3, tetrabranched NT(8-13)-PEG-6-mercaptopurine [NT4(8-13)-6-MP] 4, tetrabranched NT(8-13)-PEG-Combretastatin [NT4(8-13)-CBTST] 5, tetrabranched NT(8-13)-PEG-Monastrol [NT4(8-13)-Mon] 6, tetrabranched NT(8-13)-PEG-Tirapazamine [NT4(8-13)-TPZ] 7, tetrabranched NT(8-13)-PEG-DOTA [NT4(8-13)-DOTA] 8 and tetrabranched NT(8-13)-PEG-Combretastatin ether [NT4(8-13)-O—CBTST] 9, and tetrabranched NT(8-13)-PEG-Monastrol ether [NT4(8-13)-O-Mon] 10 (FIG. 1), were synthesized using Fmoc-Lys(Dde)-OH as first and β-Ala as second amino acid on Novasyn TGR resin, except from [NT4(8-13)-Fluo] 1 where the second aminoacid is Fmoc-PEG-OH instead of β-Ala. The tetramer was then built as above but with Boc-Arg(Pbf)-OH as last amino acid of the neurotensin sequence, so that the last two coupling steps occurred selectively on the side chain arm. Once the aminoacid sequence is completed the Dde protective group is removed with hydrazine and the intermediate is coupled with PEG. After Fmoc removal from PEG the compound is coupled to functional unit (W) carrying a free carboxyl group on the linker.

NT4(8-13)-FLUO 1, NT4(8-13)-CLB 2, NT4(8-13)-TPZ 7, NT4(8-13)-DOTA 8 as well as NT4(8-13)-O—CBTST 9 and NT4(8-13)-O-Mon 10 were linked to the branched carrier through an amide bond originating from the free carboxyl group present on the fluorophore or on the drug and the amine group of the peptide (FIG. 1). NT4(8-13)-5-FdU 3, NT4(8-13)-CBTST 5 and NT4(8-13)-Mon 6, on the other hand, were conjugated to the carrier peptide through a bifunctional linker that gave an amide bond on the peptide side and an ester bond on the drug side. NT(8-13)4-6-MP was conjugated to the carrier peptide through a bifunctional linker that gave an amide bond on the peptide side and an thio-enoic bond on the 6-MP side. The three conjugation arrangements gave rise to different drug-releasing patterns, i.e. NT4(8-13)-FLUO 1, NT4(8-13)-CLB 2, NT4(8-13)-TPZ 7, NT4(8-13)-DOTA 8, NT4(8-13)-O—CBTST 9 and NT4(8-13)-O-Mon 10 hardly release FLUO, CLB, TPZ, DOTA, CBTST and Mon. Whereas NT4(8-13)-5-FdU 3, NT4(8-13)-CBTST 5, NT4(8-13)-Mon 6 and NT(8-13)4-6-MP 4 easily release FdU, CBTST, Mon and 6-MP from the adduct.

Tetrabranched peptides carrying the sequence pyroELYENKPRRPYIL (SEQ ID No. 2) were synthesized as described above.

Unrelated branched peptides carrying the sequence AcDDHSVA (SEQ ID No. 4) were synthesized as described above and used for control.

### Peptide Internalization and Drug Release

The branched conjugated peptides 1-10 differ by the linker which is used for the coupling between the branched peptide and the functional unit. The linkers are here considered fast releasing or slow releasing for their ability to release the functional unit from the carrier peptide.

### Release of 5FdU, Monastrol and Combretastatin

10⁶ PANC-1 cells were incubated with NT conjugated branched peptides NT(8-13)4-5FdU 3, NT4(8-13)-CBTST 5, NT4(8-13)-O—CBTST 9, NT4(8-13)-Mon 6 and NT4(8-13)-O-Mon 10 (100 μM) and with the unrelated branched peptide (100 μM), at 37° C. for different time intervals. Cells were centrifuged, washed and then lysed in water after freezing and thawing. Supernatant and lysed cells were analysed by mass spectrometry after addition of NT(8-13)4 as internal standard, using an Ettan MALDI-Tof mass spectrometer in reflectron mode with an acceleration voltage of 20 kV.

NT4(8-13)-O—CBTST 9 and NT4(8-13)-O-Mon 10 decreased gradually in cell medium while increasing in cell lysate, where it appeared after 1 h, reaching maximum concentration after 48 hours of incubation. The unrelated tetra-branched peptide was found intact after 48 hours in the cell medium and never detected in the lysed cells. NT(8-13)4-5FdU decreased gradually in cell lysate and cell medium. The unrelated tetrabranched peptide conjugated to 5FdU [(AcDDHSVA)4-5FdU] was never found in the cell lysate and remained intact in the medium for 4 hours. In fact, NT(8-13)4-5FdU and (AcDDHSVA)4-5FdU are challenged by hydrolyses of 5-FdU, which is released from the ester linker, therefore they show a shorter half-life.

NT4(8-13)-CBTST 5, NT4(8-13)-Mon 6 released the CBSTS and MON after 2 hours by hydrolysis of ester bond, while ether conjugated drugs 9 and 10 were found intact in the supernatant or in lysate.

Release of 6-MP from NT(8-13)4-6-MP NT(8-13)4-6-MP was incubated at 37° C. in a phosphate buffer solution (pH=7.4) in the presence of 1, 5 and 15 equivalents of GSH. The crude mixture was then injected in HPLC at different time intervals to measured 6-MP release. NT(8-13)4-6-MP released 86% of 6-MP after 135 min in the presence of 1 equivalent of GSH and 100% release after 30 min with 5 equivalents of GSH.

The functionalized branched peptides were then classify as fast releasing or slow/non releasing: NT4(8-13)-Fluo 1, NT4(8-13)-CLB 2, NT4(8-13)-TPZ 7, NT4(8-13)-DOTA 8, NT4(8-13)-O—CBTST 9 and NT4(8-13)-O-Mon 10 are slow releasing adducts while NT4(8-13)-5-FdU 3, NT4(8-13)-CBTST 5, NT4(8-13)-Mon 6 and NT4(8-13)-6-MP 4 are fast releasing.

### In Vitro Activity of Branched NT Peptides Conjugated to Functional Unit

### Peptide Binding and Internalization in Human Cancer Cell Lines

Peptide binding and internalization, of tetra-branched NT(8-13) conjugated to Biotin (NT(8-13)4-PEG-Biotin) was analysed by confocal microscopy in human colon adenocarcinoma (HT29), human pancreas carcinoma (Panc-1) and human prostate carcinoma (PC3) cell lines¹⁵.

It was found in the present invention that NT(1-13)4-Fluorescein and NT(8-13)4-Fluorescein specifically bind to the three cell lines, which express NT receptors (FIG. 2). Cells were plated, grown for 24 hours, blocked for 30 min at −37° C. with 3% BSA in TBS and then incubated with the peptides (2 μM in TBS—0.3% BSA) compared with a four-fold molar excess of monomeric analogues. Protease inhibitors cocktail was added to the buffer in experiments with monomeric peptides. Cells were grown in the medium for 1, 2 or 4 hours at 37° C. and then were fixed with 4% formalin and plasma membrane was stained with Lectin-Cy3 (0.5 μg/ml in TBS—0.3% BSA) and nuclei with 4,6-diamidino-2-phenylindole (DAPI) (1 μg/ml in TBS—1% BSA). Images were taken by confocal laser microscope (Leica TCS SP5).

Internalization of conjugated peptides was completed in 1 hour. Peptides were degraded inside the cells within 1.8 hours¹⁵. No difference in cell binding or internalization rate was detected between NT(1-13) and NT(8-13) tetra-branched peptides. Monomeric NT(1-13)-Fluorescein (M) gave no signal.

The ability of tetrabranched peptides conjugated to a functional unit to bind cancer cell lines through NT receptors, to be rapidly internalized in cells and to still be detectable after 4 hours, show their importance for therapeutic applications.

Cytotoxicity of drug-Conjugated NT4 in Different Tumor Cell Lines

It is well known that classical chemotherapeutics used in the clinical practice have different activity on different tumors. This is due to natural resistance of cancer cells to the drugs, caused by different mechanisms, including a decreased uptake or increased export of drugs by the cell, increased inactivation of drugs inside the cell or enhanced repair of the DNA damage produced by DNA-alkylating agents.

Previously reported cytotoxicity experiments, performed by the authors of the present invention on HT-29¹⁵, demonstrated that conjugation of methotrexate (MTX) or of the photosensitizer, chlorine e6, to tetra-branched. NT(8-13) produces pro-drugs like molecules. Such molecules can no longer be transported across plasma membranes by the mechanism of the corresponding free drug and can only be ‘activated’ via peptide-receptor binding, thus profoundly decreasing non-specific drug toxicity.

Introduction of a novel, peptide receptor-mediated, mechanism of cell internalization of the drug might allow by-passing natural mechanism of cell resistance. The authors then chose several different molecules, commonly used in classical tumor chemotherapy-methotrexate (MTX, chlorarnbucil (CLB), 6-mercaptopurine and 5-fluoro-2′-deoxyuridine (5-FdU)—or emerging new drugs—combretastatin (CBTST)¹⁶, monastrol (MON)¹⁷, tirapazaminc (TPZ)¹⁸. The molecules were tested either as free drugs or conjugated to tetra-branched on HT-29, PANC-1 and PC-3 tumor cell lines (FIGS. 3 and 4). In details, HT-29, PANC-1 or PC-3 cells were plated at a density of 2.5×10⁴ per well in 96-well microplates. Different concentrations of free or NT-conjugated drugs, from 0.15 to 30 μmol/L, were added 24 h after plating. Cells were grown without changing the medium for days. Growth inhibition was assessed by 3-(4,5-dimethylthiazol-2-yl)-2,5-diphenyltetrazolium bromide. EC50 values were calculated by non-linear regression analysis using GraphPaD prism 3.02 software. The best EC50 values (expressed in molar concentrations) obtained were: 1.9e-006 for NT(8-13)4-CLB on HT29, 3.3e-007 for NT(8-13)4-5FdU on HT29, 1.4e-7 for NT(8-13)-TPZ on. PC3, 43e-007 for NT(8-13)4-CBTST on PANC-1 and 1.1e-007 for NT(1-13)4-5FdU on HT29.

The cellular toxicity of all the drug-conjugated NT4 was tested on the three cell lines and compared with the cytotoxicity of corresponding free drugs and with that of an unrelated tetra-branched peptide (U4), identically conjugated to the same drug.

5-FdU, Monastrol and CBTST were fast released from the adducts. Cytotoxicity of fast releasing drug-conjugated tetra-branched peptides was then tested in HT-29, PANC-1 and PC-3 in experiments where cells were exposed to a 1 hour pulse of free or NT-conjugated drug, washed and incubated for 6 days or, alternatively incubated for 6 days with the peptides, without additional washing. The additional washing was performed in order to avoid free drug to diffuse inside the cells during the following six days.

Conjugation to tetrabranched NT(8-13) profoundly modified drug activity, which might result from the combination of both cell and drug features, including: i) cell sensitivity to the drug; ii) drug mechanism of action; iii) mechanisms of cell resistance to the drug; iv) efficiency of membrane transport of the conjugated-drug.

As expected, activities of the free drugs are very different from one another and from cell line to cell line (FIGS. 3 and 4, second panel from the left). In principle, the conjugation to tetrabranched NT may produce as a result:
an increase in drug specificity,
an increase in drug activity,
an increase in both specificity and activity or no improvement of the free drug.

In some cases, like MTX and CLB in PC-3, conjugation to branched NT can by-pass natural cell resistance to the drug (FIG. 3) switching the cells from completely non-sensitive to full responsive. An undoubted advantage of the branched peptide carrier is its target specificity, demonstrated by the lack of activity on the three cell lines of any drug; when coupled to an unrelated branched peptide (see all third panels from left FIGS. 3 and 4). The tetrabranched NT(8-13)-PEG-6-mercaptopurine [NT4(8-13)-6-MP] 4 and tetrabranched NT(8-13)-PEG-Monastrol [NT4(8-13)-Mon]] 6 gained no improvement when compared to the parent free drugs. Results with fast releasing tetra-branched NT are very interesting, since both in the case of 5-FdU and CBTST, activity of the drug is clearly increased by conjugation to branched NT4 (FIG. 4 Panels F and G). Comparison of results obtained with CBTST in slow and fast releasing molecules, indicate that fast releasing molecules can be even more active than slow-releasing compounds.

Interestingly, cell that are not affected by a drug; such as PC-3 by MTX, CLB and 5-FdU, can become sensitive to it, when conjugated to branched NT (FIG. 4 Panels A, B and F). Changing the mechanism of membrane transport, by switching to a peptide receptor-mediated mechanism, can deeply modify drug transport from outside to inside the cells. Moreover, conjugation to branched peptides might as well impair mechanisms of drug export from inside to outside the cell, entrapping the conjugated drug into the target cell. This is extremely important for the therapy of tumors that over-express NT receptors and 1.0 do not respond to classical chemotherapy.

Analysis of Human Tumor Samples from Surgical Resections Using Fluorophore-Conjugated NT4

In order to validate the NT branched peptides of the present invention as possible targeting agents for therapy of either colon or pancreas adenocarcinoma, binding of tetra-branched NT peptides to human tumor surgical samples in comparison to healthy tissues, was analysed and quantified. Surgical resections of 16 colon and 12 pancreas tumors were collected. Tumor samples were compared to healthy tissues from the same patient obtained 5 cm away from the tumor edge. Serial sections of the same biopsy were analysed both by hematoxylin/eosin (H & E) light microscopy and by fluorescent confocal microscopy. In details, samples were embedded in tissue tck and stored in liquid nitrogen. 10 μm thick sections, obtained with a 2800 Frigocut N (Reichert-Jung, Depew, N.Y.), were dried at 37° C. for 30 min, fixed with 4% formalin for 15 min at room temperature and incubated in glycine 0.1M for 12 hours at 4° C. Blocking with FBS for 30 min at 37° C. was followed by 30 min incubation at room temperature either with NT(1-13)4-Fluorescein (1 μg/ml in TBS—0.3% BSA). Each step was followed by washing with TBS. Finally, sections were incubated for 0.5 min with 4,6-diamidino-2-phenylindole (DAPI) (1 μg/ml in TBS—1% BSA). Each step was followed by washing with TBS. Controls were performed using an unrelated Fluorescein-conjugated tetra-branched peptide. Analogue monomeric peptides were assayed for comparison. Peptide binding was analyzed by confocal laser microscope (Leica TCS SP5) with 488 nm absorption and 500-540 emission wavelength for Fluorescein and 405 absorption and 420-460 emission for DAPI. All images were processed using the ImageJ software (NIH). Resulting electronic data were reported as pixel distribution in the green color range of the RGB system.

This enabled translation of the immunofluorescence signals of tumor and healthy tissues into numbers representing the mean of green staining in the range of the RGB system, for each sample (FIG. 5 Panel B).

When treated with NT(1-13)4-Fluorescein tumor tissues from both colon and pancreas adenocarcinoma showed remarkably higher fluorescence emission compared to normal tissues from the same patients (FIG. 5 Panel A). Binding of NT(1-13)4 to any tissue sample was identical to that of NT(8-13)4 to the same sample.

14 out of the 16 colon cancers were histologically characterized as adenocarcinomas and 2 as adenomas. The latter had K/H values corresponding to the lowest range of the K/H ranking. For pancreas samples, 11 out of the 12 samples were adenocarcinomas and one was a lymphoma, ie it had a very different cell origin. This sample had the lowest K/H of all tested surgical samples. No correlation was found between staging of the tumors, either colon or pancreas, and receptor expression (K/H value). This means that even at early stages of the disease the difference between K and H tissues is statistically relevant. This is a very important point for a tumor marker that is not uniquely expressed by tumor cells but rather overexpressed by them. NT receptors might then be used as targets for early treatments with branched peptides (Table 1, FIG. 6)

TABLE 1
Clinical features of colon and pancreas tumor samples.

| | | | | | | |
|---|---|---|---|---|---|---|
| entry | K/H | gender | age | type | grading | TNM |

COLON CARCINOMA

| | | | | | | |
|---|---|---|---|---|---|---|
| 1 | 2.8 | M | 64 | NOS adenocarcinoma | g2 | T2N0Mx |
| 2 | 3.8 | M | 70 | NOS adenocarcinoma | g2 | T3N1Mx |
| 3 | 3.5 | F | 63 | NOS adenocarcinoma | g2 | T3N0Mx |
| 4 | 1.8 | M | 69 | NOS adenocarcinoma | g2 | T3N2Mx |
| 5 | 0.8 | M | 79 | NOS adenocarcinoma | g2 | T3N0Mx |
| 6 | 2.2 | M | 77 | NOS adenocarcinoma | g3 | T4N0Mx |
| 7 | 2.1 | M | 66 | NOS adenocarcinoma | g2 | T4N0Mx |
| 8 | 2.2 | M | 75 | NOS adenocarcinoma | g3 | T4n1M1 |
| 9 | 2.9 | F | 66 | NOS adenocarcinoma | g2 | T2N0Mx |
| 10 | 1.6 | M | 55 | adenoma | — | — |
| 11 | 2.3 | F | 75 | mucinous | g2 | T3N0Mx |
| | | | | adenocarcinoma | | |
| 12 | 2.3 | M | 71 | NOS adenocarcinoma | g4 | T4N2M1 |
| 13 | 2.0 | F | 79 | NOS adenocarcinoma | g3 | T3N0Mx |
| 14 | 2.1 | F | 75 | mucinous | g1 | T3N0x |
| | | | | adenocarcinoma | | |
| 15 | 5.7 | F | 83 | NOS adenocarcinoma | g2 | T3N2Mx |
| 16 | 0.8 | M | 73 | adenoma | — | — |

PANCREAS CARCINOMA

| | | | | | | |
|---|---|---|---|---|---|---|
| 1 | 1.7 | F | 54 | NOS adenocarcinoma | g2 | T1N0Mx |
| 2 | 2.4 | F | 69 | ductal adenocarcinoma | g2 | T2N0Mx |
| 3 | 2.4 | M | 68 | ductal adenocarcinoma | g2 | T4n1Mx |
| 4 | 7.5 | F | 69 | NOS adenocarcinoma | g1 | T3N0Mx |
| 5 | 2.1 | F | 64 | NOS adenocarcinoma | g2 | T3N1Mx |
| 6 | 4.1 | F | 64 | ductal adenocarcinoma | g2 | T3N1Mx |
| 7 | 2.2 | M | 74 | ductal adenocarcinoma | g2 | T3N0Mx |
| 8 | 5.4 | M | 72 | adenocarcinoma | g2 | T2N0Mx |
| 9 | 3.2 | F | 47 | pseudopapillary | / | T2n0Mx |
| | | | | carcinoma | | |
| 10 | 2.8 | M | 55 | ductal adenocarcinoma | g3 | T4N1Mx |
| 11 | 2.6 | M | 68 | ductal adenocarcinoma | g2 | T3N1Mx |
| 12 | 1.6 | M | 64 | linfoma | — | — |

K/H was calculated on the average values of K and H RGB values.
Legend: NOS: not otherwise specified. Grading = tumor grading on the basis of cytology observations. TNM (international staging of tumors): T = tumor size; N = number of lymph nodes involved, M = number of metastasis.

Statistical analysis was performed to evaluate significance of difference in peptide binding between healthy and tumor tissues from all collected surgical samples, except for the lymphoma because of its completely different cell origin, with respect to colon and pancreas carcinomas. As shown in the box-plots (FIG. 6), a remarkable difference in signal was observed for both pancreas and colon cancers, with respect to their healthy counterparts. For the comparison between healthy and tumor samples, the level of significance was p<0.01 for pancreas and p<0.02 for colon for two-sided testing. This result means that these peptides can discriminate between healthy and cancer samples, therefore they can be used as tumor markers.

This result is very promising for possible therapy of colon carcinoma and pancreas exocrine carcinoma by means of NT-branched peptides. Imaging of tumor biopsies might enable pre-treatment estimation of the efficacy of a NT-based target therapy, which might be evaluated on the basis of measured differences of branched peptide binding in tumor versus healthy tissue, in each patient. Moreover, such a clear discriminating signal between healthy and tumor tissues indicates that branched NT might play a remarkable role in the specific diagnosis of colon and pancreas carcinoma.

In Vivo Activity of branched NT Peptides Conjugated to Functional Unit.

### Tumor Growth Reduction in Mice

Nude mice bearing HT29 tumors in the right flank were injected with six doses of NT(1-13)-5FdU. Compound NT(1-13)-5FdU was chosen for the in vivo experiments among all, in the light of its in vitro cytotoxicity on HT29, when compared to the analogue peptides coupled to MTX.

In details, CD-1 female nude mice (Charles River Laboratories, Inc.), 5 to 6 weeks of age (mean weight, 20 g), were injected s.c. in the right flank with 1×10⁶ HT29 cells. When tumors reached a diameter of 3 to 4 mm (5 days after tumor inoculation) mice were randomly divided into groups (four mice per group) and repeatedly injected in the tail vein (0, 30, 70, 140, 190, 240 hours post first injection) with 500 μL of the following solutions in 0.9% NaCl: (a) 1 mg/mL NT(1-13)4-5FdU (3.05 μmol/kg); (b) 30 μg/mL 5FdU (3.05 μmol/kg); and (c) 0.9% NaCl.

Tumor volumes were measured daily with a caliper using the following formula: volume=length*width2*π/6. 21 days after tumor inoculation, ie 6 days after the last treatment, mice were sacrificed, tumors, removed and weighted. Experiments were done following the local ethical Committee approval for animal use in cancer research. The procedures related to animal use conform to all regulations protecting animals used for research purposes, including UKCCCR (1998) United Kingdom Co-ordinating Committee on Cancer Research guidelines for the welfare of animals in experimental neoplasia. 2nd ed Br J Cancer 77: 1-10. Statistical analysis was done using Student's t test (p<0.05). After the last treatment (240 h), the average tumor volume of the mice treated with NT(1-13)-5FdU was around 50% that of animals treated with free 5FdU or with saline (FIG. 7A). Tumor weight, measured 5 days after the last treatment (360 h), was 40% less in mice treated with the drug conjugated peptide than in the control groups (FIG. 7C). Free 5-FdU given six times, in a dose of 3 μg/kg, in time-frame of 10 days, gave only 10% inhibition of tumor growth.

### BIBLIOGRAPHY

1. Reubi J C. J Nucl Med. 1995; 36(10): 1825-35.
2. Reubi J C. Endocr Rev. 2003; 24(4): 389-427.
3. Langer M and Beck-Sickinger A G. Curr Med Chem Anti-Canc Agents. 2001; 1(1): 71-93.
4. Garcia-Garayoa E, Allemann-Tannahill L, Blauenstein P, Willmann M, Carrel-Remy N, Tourwe D, Iterbeke K, Conrath P, Schubiger P A. Nucl Med Biol. 2001; 28(1): 75-84.
5. Hillairet De Boisferon M, Raguin O, Thiercelin C, Dussaillant M, Rostene W, Barbet J, Pelegrin A, Gruaz-Guyon A. Bioconjug Chem. 2002; 13(3): 654-62.
6. Achilefu S, Srinivasan A, Schmidt M A, Jimenez H N, Bugaj J E, Erion J L. J Med Chem. 2003; 46(15): 3403-11.
7. Bergmann R, Scheunemann M, Heichert C, Mading P, Wittrisch H, Kretzschmar M, Rodig H, Tourwe D, Iterbeke K, Chavatte K, Zips D, Reubi J C, Johannsen B. Nucl Med Biol. 2002; 29(1): 61-72.
8. de Visser M, Janssen P J J M, Srinivasan A, Reubi J C, Waser B, Erion J L, Schmidt M A, Krenning E P, de Jong M. Eur J Nucl Med Mol Imaging. 2003; 30(8): 1134-9.
9. Buchegger F, Bonvin F, Kosinski M, Andreas O, Schaffland A O, Prior J, Reubi J C, Bläuenstein P, Tourwé D, Garcia Garayoa E and Bischof Delaloye A. J Nucl Med. 2003; 44(10): 1649-54.
10. Morinville A, Martin S, Lavallee M, Vincent J P, Beaudet A, Mazella J. Int J Biochem Cell Biol. 2004; 36(11): 2153-68.
11. Reubi J C, Mäcke H R, Krenning E P. J Nucl Med. 2005; 46: 67S-75S
12. Reubi J C, Waser B, Friess H, Buehler M, Laissue J. Gut. 1998; 42(4):546-550
13. Bracci L, Falciani C, Lelli B, Lozzi L, Runci Y, Pini A, De Montis M G, Tagliamonte A, Neri P. J Biol Chem. 2003; 278(47): 46590-5.
14. Falciani C, Lozzi L, Pini A, Corti F, Fabbrini M, Bernini A, Lelli B, Niccolai N, Bracci L. Chem Biol Drug Des. 2007, 69:216-21.
15. Falciani C, Fabbrini M, Pini A, Lozzi L, Lelli B, Pileri S, Brunetti J, Bindi S, Scali S, Bracci L. Mol Cancer Ther. 2007; 6:2441-8.
16. Gwaltney S L 2nd, Invade H M, Li Q, Gehrke L, Credo R B, Warner R B, Lee J Y, Kovar P, Frost D, Ng S C, Sham H L. Novel sulfonate derivatives: potent antimitotic agents. Bioorg Med Chem Lett. 2001; 11:1671-3.
17. Lad L, Luo L, Carson J D, Wood K W, Hartman J J, Copeland R A, Sakowicz R. Mechanism of inhibition of human KSP by ispinesib, Biochemistry. 2008 Mar. 18; 47(11):3576-85.
18. Bache M, Kappler M, Said H M, Staab A, Vordermark D. Curr Med Chem. 2008; 15:322-38.

1. A multimeric molecule having the general formula A or B: 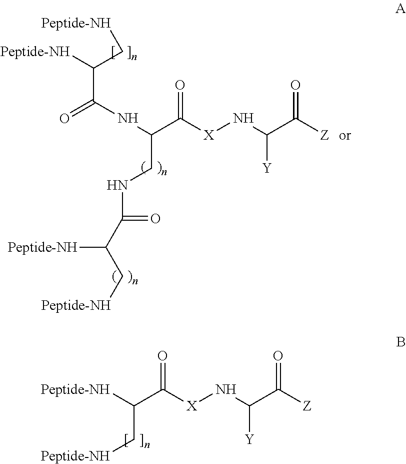
wherein
n=1-5, 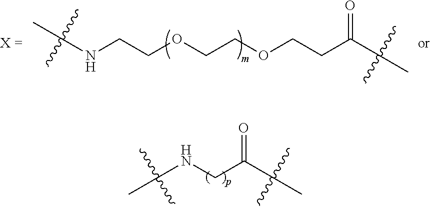
wherein m=9-12 and p=1-5, 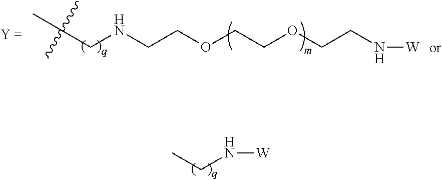
wherein q=1-5 and m=9-12; 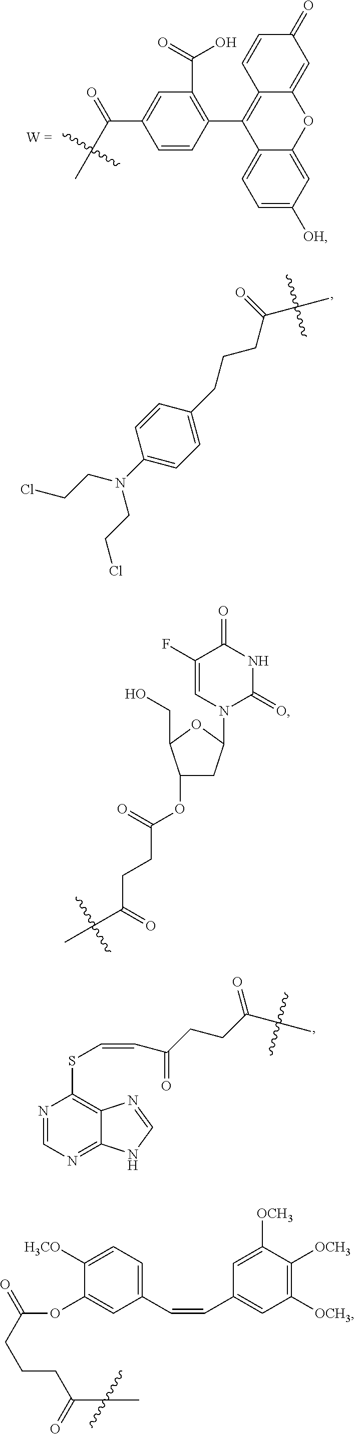 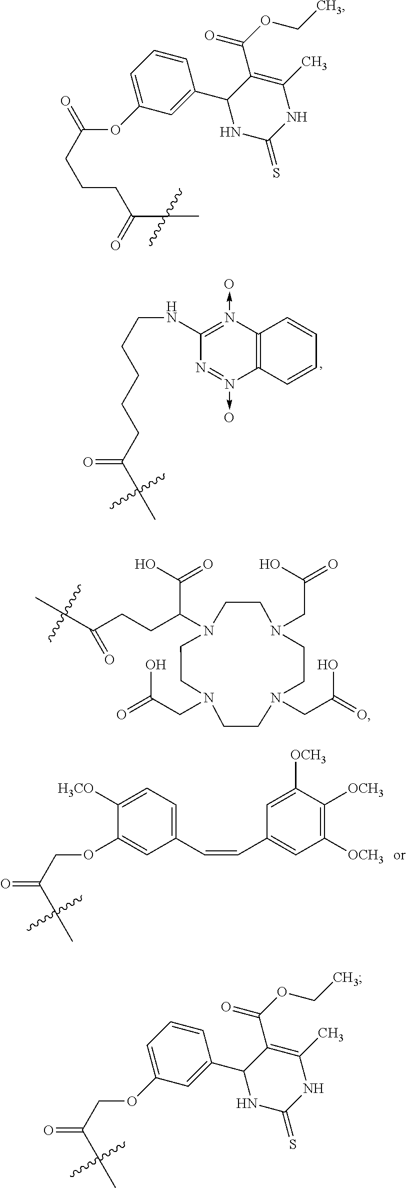
and Z=NH₂ or OH.

2. The multimeric molecule according to claim 1, wherein the peptide comprises the following amino acid sequence: QLYENKPRRPYIL (SEQ ID No. 1), pyroEL YENKPRRPYIL (SEQ ID NO. 2) or RRPYIL (SEQ ID No 5, a.a. 8 to a.a. 13 of SEQ ID No. 1).

3-9. (canceled)

10. A pharmaceutical composition comprising the multimeric molecule according to claim 1, or a pharmaceutically acceptable and efficient salt thereof and diluents, and/or solvents and/or carriers and/or excipients.

11. A method of treatment comprising the administration or exposure to the multimeric molecule according to claim 1.

12. The method according to claim 11, wherein the treatment is anti-tumoral.

13. A method of diagnosis comprising the administration or exposure to the multimeric molecule according to claim 1.

14. The method according to claim 13, wherein the diagnosis is a tumor diagnosis.

15. The method according to claim 12, wherein the tumor is a colon or pancreas or prostate carcinoma.

16. A method of treating a colon, or pancreas or prostate carcinoma in a patient in need thereof comprising administering an effective amount multimeric molecule according to claim 1.

17. A method of preparing a medicament with the multimeric molecule according to claim 1.

18. The method according to claim 17, wherein the medicament has an anti-tumoral activity.

19. A method of diagnosing a tumor with the multimeric molecule according to claim 1.

20. The method according to claim 18, wherein the tumor is a colon or pancreas or prostate carcinoma.
